(19) United States
(12) Patent Application Publication
(10) Pub. No.: US 2022/0322939 A1
(43) Pub. Date: Oct. 13, 2022

(54) HEALTH MONITORING METHOD AND SYSTEM

(71) Applicant: SENSOMICS, INC., Burlingame, CA (US)

(72) Inventors: Lichen ZHU, Burlingame, CA (US); Sixing LIU, Burlingame, CA (US); Jingjing LI, Burlingame, CA (US); Zhaolei ZHANG, Burlingame, CA (US)

(21) Appl. No.: 17/838,279

(22) Filed: Jun. 13, 2022

Related U.S. Application Data
(63) Continuation of application No. PCT/CN2019/126475, filed on Dec. 19, 2019.

(51) Int. Cl. A61B 5/00; G16H 10/60

(52) U.S. Cl. CPC A61B 5/0022; A61B 5/7475; A61B 5/746; A61B 5/743; A61B 5/7203; G16H 10/60

(57) ABSTRACT

A health monitoring method, including: acquiring physiological parameters of a user in real time; storing the physiological parameters and establishing a user health model according to the stored physiological parameters, the user health model including indicators of physiological parameters of the user in a healthy state; and determining the level of the current physiological parameters of the user according to the indicators of the physiological parameters of the user health model and determining the physiological state of the user. In the method, physiological parameters of the user are monitored by means of the user health model. If the current physiological parameters of the user exceed a range of the indicators of the physiological parameters, then prompt information of physiological parameters being abnormal is generated, enabling the user to learn which physiological parameter is abnormal, thereby facilitating the user in rapidly discovering a problem, and responding more quickly.

### CROSS-REFERENCE TO RELATED APPLICATION

This application is a continuation of international application No. PCT/CN2019/126475 filed on Dec. 19, 2019, the content of which is hereby incorporated by reference in its entirety.

### TECHNICAL FIELD

The present disclosure generally relates to a technical field of physiological parameter detection, and in particular, to a health monitoring method and a system.

### BACKGROUND

The development of technologies in detecting physiological parameters have contributed to more types of wearable devices, especially sport devices, which can record a user's movement. Conventional devices are mainly used for movement display, usually displaying information such as heart rate, a step count, or a calorie consumption calculated from the heart rate or the step count. Current monitoring methods do not summarize these data or information to build a user health model, and merely simply record the data or information when the data or information is abnormal.

### SUMMARY

It is desired to provide a health monitoring method and a system.

A health monitoring method includes: acquiring physiological parameters of a user in real time, storing the physiological parameters, establishing a user health model including indicators of physiological parameters of the user in a healthy state according to the stored physiological parameters, determining a level of current physiological parameters of the user according to the indicators of the physiological parameters of the user health model, and determining a physiological state of the user.

In an example of the present disclosure, before the acquiring physiological parameters of the user in real time, the method further includes: obtaining identification information of the user, and verifying whether the identification information of the user is consistent with identification information pre-stored in a database, upon a condition that the identification information of the user is consistent with identification information pre-stored in the database, acquiring the physiological parameters of the user, and upon a condition that the identification information of the user is not consistent with identification information pre-stored in the database, creating new identification information of the user and acquiring the physiological parameters of the user.

In an example of the present disclosure, the acquiring physiological parameters of the user in real time includes pre-processing of the physiological parameters and removing abnormal physiological parameters.

In an example of the present disclosure, the pre-processing of the physiological parameters includes smoothing and normalizing the physiological parameters.

In an example of the present disclosure, the method further includes periodically updating the user health model according to the acquired physiological parameters of the user to obtain updated indicators of physiological parameters of the user in the healthy state.

In an example of the present disclosure, the physiological parameters include at least one of a pulse rate, a skin temperature, a skin resistance, or a step count.

In an example of the present disclosure, the determining the level of current physiological parameters of the user according to the indicators of the physiological parameter of the user health model and determining the physiological state of the user includes: upon a condition that the current physiological parameters of the user exceed a range of the indicators of the physiological parameters, generating prompt information of physiological parameters being abnormal.

A health monitoring system includes: a physiological parameter acquiring unit configured for acquiring physiological parameters of a user in real time; and a processing unit configured for storing the physiological parameters, establishing a user health model including indicators of physiological parameters of the user in a healthy state according to the stored physiological parameters, determining a level of current physiological parameters of the user according to the indicators of the physiological parameters of the user health model, and determining a physiological state of the user.

In an example of the present disclosure, the physiological parameter acquiring unit includes a wearable device, and/or the processing unit comprises a server.

In an example of the present disclosure, the processing unit is further configured for transmitting information of a current level of physiological parameters of the user and physical status of the user to a user terminal.

### BRIEF DESCRIPTION OF THE DRAWINGS

To describe and illustrate embodiments and/or examples of the present disclosure made public here better, reference may be made to one or more of the figures. The additional details or examples used to describe the figures should not be construed as limiting the scope of any of the present disclosure, the embodiments and/or examples currently described, and the best model of the present disclosure as currently understood.

FIG. 1 is a diagram of an application environment for a health monitoring method according to an example of the present disclosure.

FIG. 2 is a schematic diagram of an operation of a health monitoring method according to an example of the present disclosure.

FIG. 3 is a flowchart diagram of a health monitoring method according to an example of the present disclosure.

FIG. 4 is a block diagram of a structure of a health monitoring system according to an example of the present disclosure.

FIG. 5 is a diagram of an internal structure of a computer device according to an example of the present disclosure.

FIG. 6 is a diagram of an internal structure of a computer device according to another example of the present disclosure.

In the figures, 102 represents a terminal; 104 represents a server; 106 represents a health monitoring device; 510 represents a physiological parameter acquiring unit; 520 represents a processing unit; 530 represents an identification information verifying unit.

### DETAILED DESCRIPTION OF THE EMBODIMENT

The technical solutions in the examples of the present disclosure are clearly and completely described in the following with reference to the accompanying drawings in the examples of the present disclosure. It is obvious that the described examples are only a part of the examples, but not all of the examples. All other examples obtained by those skilled in the art based on the examples of the present disclosure without making creative labor are the scope of the present disclosure.

Unless otherwise defined, all technical and scientific terms used herein have the same meaning as a skilled person in the art would understand. The terminology used in the description of the present disclosure is for the purpose of describing particular examples and is not intended to limit the disclosure. The term “or/and” as used herein includes any and all combinations of one or more of the associated listed items.

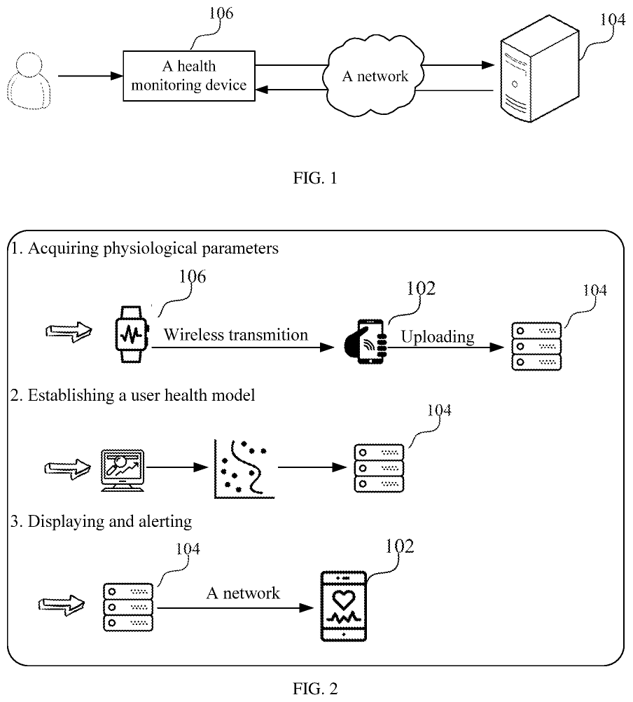

A health monitoring method provided in the present disclosure can be applied to an application environment as shown in FIG. 1. A health monitoring device 106 can communicate with a server 104 via a network. The health monitoring device 106 can acquire physiological parameters of a user and transmit acquired physiological parameters to the server 104. The server 104 can receive and store the physiological parameters, and establish a user health model according to the stored historical physiological parameters. The user health model can include indicators of physiological parameters of the user in a healthy state. A level of current physiological parameters of the user can be determined according to the indicators of the physiological parameters of the user health model, and a physiological state of the user can be determined.

A health monitoring method provided in the present disclosure can be further applied to an application environment as shown in FIG. 2. The health monitoring device 106 can communicate with a terminal 102 via a Bluetooth, and the terminal 102 can communicate with the server 104 via a network. The health monitoring device 106 can acquire physiological parameters of the user and transmit acquired physiological parameters to the terminal 102. The terminal 102 can transmit the physiological parameters to the server 104. The server 104 can receive and store the physiological parameters, and establish a user health model according to the stored historical physiological parameters. The user health model can include indicators of physiological parameters of the user in the healthy state. A level of current physiological parameters of the user can be determined according to the indicators of the physiological parameters of the user health model, and a physiological state of the user can be determined. The server 104 can transmit the user health model to the terminal 102 and update information in the user health model in real time, enabling the user to learn change of the physiological parameters in the first place, thereby facilitating the user in rapidly discovering a problem and responding more quickly. The terminal 102 can be, but is not limited to, a variety of personal computers, laptops, smartphones, tablets, or portable wearable devices. The server 104 can be an independent server or a server cluster of multiple servers. The terminal 102 can be an independent terminal or a plurality of terminals. Alternatively, the terminal 102 can be a computer device with functions of Bluetooth and wireless communication.

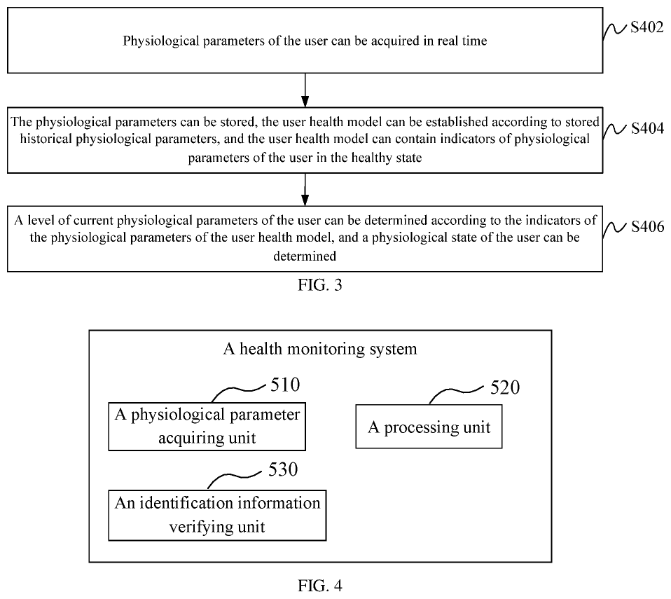

In an example, as shown in FIG. 3, a health monitoring method is provided. An application of the method in an application environment in FIG. 1 is illustrated as an example. The method includes the following steps.

At step 402, physiological parameters of the user can be acquired in real time.

The physiological parameters can include at least one of a pulse rate, a skin temperature, a skin resistance, or a step count.

Specifically, at step 402, the user can use the health monitoring device 106 to acquire the physiological parameters of the user, and transmit the physiological parameters to the server 104. It can be understood that the health monitoring device 106 can be a wearing device, a handheld device or other device that can acquire the physiological parameters of the user in real time.

Step 402 can further include pre-processing of the physiological parameters and removing abnormal physiological parameters. The pre-processing can include normalization, smoothing, erasing abnormal data, or other means. In an example, the server 104 can smooth and normalize the physiological parameters.

At step 404, the physiological parameters can be stored, the user health model can be established according to the stored historical physiological parameters, and the user health model can include indicators of physiological parameters of the user in the healthy state.

The server 104 can receive and store the physiological parameters, and establish the user health model according to the stored historical physiological parameters. The user health model can includes indicators of physiological parameters of the user in the healthy state.

Step 404 can further include periodically updating the user health model according to the acquired physiological parameters of the user to obtain updated indicators of physiological parameters of the user in the healthy state. The healthy state of the user can be monitored accurately by constantly updating the indicators of the physiological parameters.

In an example, the user health model can identify data that is no longer within a range of the indicators of the physiological parameters in a conspicuous manner. In an example, the user health model can identify in a color gradient from the middle value of the indicators of the physiological parameters to outside the indicators of the physiological parameters, enabling the user to clearly and intuitively view dynamic changes in the physiological parameters, view historical physiological parameters, and learn about physiological parameter changes.

At step 406, a level of current physiological parameters of the user can be determined according to the indicators of the physiological parameters of the user health model, and a physiological state of the user can be determined.

Step 406 can further include upon a condition that the current physiological parameters of the user exceed a range of the indicators of the physiological parameters, generating prompt information of physiological parameters being abnormal. In an example, the server 104 can transmit the prompt information to the terminal 102, and the terminal 102 can prompt the user that the current physiological parameters exceed a range of the indicators of the physiological parameters. In another example, the server 104 can transmit the prompt information to the health monitoring device 106, and the health monitoring device 106 can prompt the user that the current physiological parameters exceed a range of the indicators of the physiological parameters. The prompt information can facilitate the user in rapidly discovering the problem, and responding more quickly.

The health monitoring method further includes: obtaining identification information of the user, and verifying whether the identification information of the user is consistent with identification information pre-stored in a database, upon a condition that the identification information of the user is consistent with identification information pre-stored in the database, acquiring the physiological parameters of the user, and upon a condition that the identification information of the user is not consistent with identification information pre-stored in the database, creating new identification information of the user and acquiring the physiological parameters of the user.

Specifically, upon the condition that the identification information of the user is consistent with identification information pre-stored in the database, the user health model corresponding to the identification information of the user in the database can be extracted. The abnormal physiological parameters can be detected more quickly, without the need to re-establish the user health model, and simply by adjusting the user health model and/or the indicators of the physiological parameters according to the physiological parameters acquired in real time. Upon the condition that the identification information of the user is not consistent with identification information pre-stored in the database, new identification information of the user can be created, and the physiological parameters of the user can be acquired. A new user health model established according to the physiological parameters can be stored in the database of the server 104 corresponding to the new identification information of the user. The new user health model can be directly called when used again.

The health monitoring method can further include transmitting the user health model to the terminal 102.

Specifically, the server 104 can transmit the user health model to the terminal 102.

In an example, the terminal 102 can be a plurality of terminals, the plurality of terminals all can receive the user health model and alerts. Owners of the plurality of terminals all can view the user health model. Upon a condition that the current physiological parameters of the user are abnormal, the plurality of terminals can receive prompt information of physiological parameters being abnormal, and the owners can assist the user.

Furthermore, a corresponding APP can be installed in the terminal 102. The APP can display pre-processed physiological parameters. Upon a condition that the terminal 102 is connected to the network, the user health model can be updated in real time through the server 104. Upon a condition that the terminal 102 is not connected to the network and the terminal 102 can communicate with the health monitoring device 106, the terminal 102 can update the user health model according to the physiological parameters transmitted by the health monitoring device 106.

In the above health monitoring method, the health monitoring device 106 can acquire the physiological parameters of the user in real time and transmit the physiological parameters to the server 104. The server 104 can store the physiological parameters, and establish the user health model according to the stored historical physiological parameters. The user health model can include indicators of physiological parameters of the user in the healthy state. The server 104 can transmit the user health model to the terminal 102. The terminal 102 can determine the level of current physiological parameters of the user according to the indicators of the physiological parameters of the user health model, and determine a physiological state of the user. Upon the condition that the current physiological parameters of the user exceed a range of the indicators of the physiological parameters, prompt information of physiological parameters being abnormal is generated, enabling the user to learn which physiological parameter is abnormal, thereby facilitating the user in rapidly discovering the problem and responding more quickly.

It should be understood that although the steps in the flowchart diagram in FIG. 4 are shown in sequence as indicated by arrows, the steps are not necessarily executed in the order indicated by the arrows. Unless explicitly stated in the present disclosure, there is no strict order in which the steps can be performed, and the steps can be performed in any other order. In addition, at least a part of the steps in FIG. 4 can include multiple sub-steps or stages. The sub-steps or the stages are not necessarily executed at the same time, but can be executed at different times. An execution sequence of the sub-steps or the stages is not necessarily sequential. The sub-steps or the stages may be performed alternately or in turn with other steps or at least a part of a sub-step or phase of other steps.

In an example, as shown in FIG. 4, a health monitoring system provided can include a physiological parameter acquiring unit 510 and a processing unit 520.

The physiological parameter acquiring unit 510 is configured for acquiring physiological parameters of a user in real time.

The processing unit 520 is configured for storing the physiological parameters, establishing a user health model according to the stored physiological parameters, determining a level of current physiological parameters of the user according to the indicators of the physiological parameters of the user health model, and determining a physiological state of the user. The user health model can include indicators of physiological parameters of the user in a healthy state.

In an example, the physiological parameter acquiring unit 510 can include a wearable device, and/or the processing unit 520 can include a server 104.

The processing unit 520 is further configured for pre-processing of the physiological parameters and removing abnormal physiological parameters. The pre-processing of the physiological parameters includes smoothing and normalizing the physiological parameters.

The processing unit 520 is further configured for periodically updating the user health model according to the acquired physiological parameters of the user to obtain updated indicators of physiological parameters of the user in the healthy state.

The processing unit 520 is further configured for generating prompt information of physiological parameters being abnormal upon a condition that the current physiological parameters of the user exceed a range of the indicators of the physiological parameters.

The health monitoring system can further include an identification information verifying unit 530 configured for obtaining identification information of the user, and verifying whether the identification information of the user is consistent with identification information pre-stored in a database, upon a condition that the identification information of the user is consistent with identification information pre-stored in the database, acquiring the physiological parameters of the user, and upon a condition that the identification information of the user is not consistent with identification information pre-stored in the database, creating new identification information of the user and acquiring the physiological parameters of the user.

Specifically, upon the condition that the identification information of the user is consistent with identification information pre-stored in the database, the user health model corresponding to the identification information of the user in the database can be extracted. The abnormal physiological parameters can be detected more quickly, without the need to re-establish the user health model, and simply by adjusting the user health model and/or the indicators of the physiological parameters according to the physiological parameters acquired in real time. Upon the condition that the identification information of the user is not consistent with identification information pre-stored in the database, new identification information of the user can be created, and the physiological parameters of the user can be acquired. A new user health model established according to the physiological parameters can be stored in the database of the server 104 corresponding to the new identification information of the user. The new user health model can be directly called when used again.

Specific limitations of the health monitoring system can be found in limitations of the health monitoring method above and would not be repeated here. Individual units in the above health monitoring system may be implemented in whole or in part by software, hardware, and combinations thereof. Each of the above units may be embedded in hardware form or independent of a processor in a computer device, or may be stored in software form in a memory in the computer device, resulting in that the processor can be called to perform operations corresponding to each of the above units.

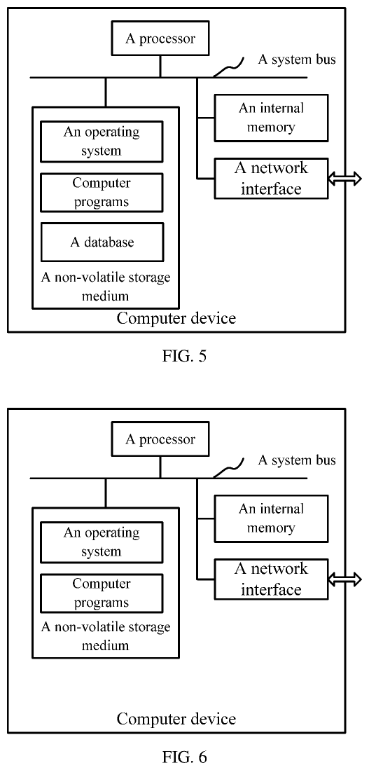

In an example, a computer device provided can be the server 104, an internal structure of which is as shown in FIG. 5. The computer device can include a processor, a memory, a network interface, and a database connected via a system bus. The processor of the computer device is configured to provide computing and control capabilities. The memory of the computer device can include a non-volatile storage medium and an internal memory. The non-volatile storage medium can store an operating system, a computer program, and a database. The internal memory can provide an environment for the operation of the operating system and the computer program in the non-volatile storage medium. The database of the computer device is configured to store the user health model as well as user data. The network interface of the computer device is configured to communicate with the terminal 102 and/or the health monitoring device 106 via a network connection. The computer program can be executed by the processor to implement a user health model establishing method.

In an example, a computer device provided can be the health monitoring device 106, an internal structure of which is as shown in FIG. 6. The computer device can include a processor, a memory, and a network interface connected via a system bus. The processor of the computer device is configured to provide computing and control capabilities. The memory of the computer device can include a non-volatile storage medium and an internal memory. The non-volatile storage medium can store an operating system and a computer program. The internal memory can provide an environment for the operation of the operating system and the computer program in the non-volatile storage medium. The network interface of the computer device is configured to communicate with the terminal 102 and/or the server 104 via a network connection. The computer program can be executed by the processor to implement the health monitoring method.

It could be understood by those of ordinary skill in the art that the structures shown in FIG. 5 and FIG. 6 are merely block diagrams of portions of the structures associated with the examples of the present disclosure and do not constitute a limitation of the computer device to which the examples of the present disclosure are applied, and that specific computer devices may include more or fewer components than those shown in the figures, combine certain components, or have different arrangements of components.

A person of ordinary skill in the art can understand that all or part of the processes in the methods of the above examples can be performed by means of a computer program to instruct the relevant hardware to do so. The computer program may be stored in a non-volatile computer readable storage medium. When the computer program is executed, processes such as those of the examples of each of the methods described above can be included. Any reference to a memory, a storage, a database, or other media used in the examples provided in the present disclosure may include non-volatile and/or volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. By way of illustration and not limitation, the RAM can be available in a variety of forms, such as a static RAM (SRAM), a dynamic RAM (DRAM), a synchronous DRAM (SDRAM), a double data rate SDRAM (DDRSDRAM), an enhanced SDRAM (ESDRAM), a synchronous link DRAM (SLDRAM), a memory bus (Rambus) direct RAM (RDRAM), a direct memory bus dynamic RAM (DRDRAM), and a memory bus dynamic RAM (RDRAM), etc.

The technical features of the above-described examples may be combined in any combination. For the sake of brevity of description, not all possible combinations of the technical features in the above examples are described. However, as long as there is no contradiction between the combinations of these technical features, all should be considered as within the scope of this disclosure.

The above-described examples are merely illustrative of several examples of the present disclosure, and the description thereof is relatively specific and detailed, but is not to be construed as limiting the scope of the disclosure. It should be noted that a number of variations and modifications may be made by those skilled in the art without departing from the spirit and scope of the disclosure. Therefore, the scope of the disclosure should be determined by the appended claims.

We claim:

1. A health monitoring method comprising:
acquiring physiological parameters of a user in real time;
storing the physiological parameters and establishing a user health model according to the stored physiological parameters, wherein the user health model comprises indicators of physiological parameters of the user in a healthy state; and
determining a level of current physiological parameters of the user according to the indicators of the physiological parameters of the user health model and determining a physiological state of the user.

2. The method of claim 1, wherein before the acquiring physiological parameters of the user in real time, the method further comprises:
obtaining identification information of the user; and
verifying whether the identification information of the user is consistent with identification information pre-stored in a database, wherein
upon a condition that the identification information of the user is consistent with identification information pre-stored in the database, acquiring the physiological parameters of the user, and
upon a condition that the identification information of the user is not consistent with identification information pre-stored in the database, creating new identification information of the user and acquiring the physiological parameters of the user.

3. The method of claim 1, wherein the acquiring physiological parameters of the user in real time comprises:
pre-processing of the physiological parameters and removing abnormal physiological parameters.

4. The method of claim 3, wherein the pre-processing of the physiological parameters comprises smoothing and normalizing the physiological parameters.

5. The method of claim 1, wherein the method further comprises:
periodically updating the user health model according to the acquired physiological parameters of the user to obtain updated indicators of physiological parameters of the user in the healthy state.

6. The method of claim 1, wherein the physiological parameters comprise at least one of a pulse rate, a skin temperature, a skin resistance, or a step count.

7. The method of claim 1, wherein the determining the level of current physiological parameters of the user according to the indicators of the physiological parameter of the user health model and determining the physiological state of the user comprises:
upon a condition that the current physiological parameters of the user exceed a range of the indicators of the physiological parameters, generating prompt information of physiological parameters being abnormal.

8. A health monitoring system comprising:
a physiological parameter acquiring unit configured for acquiring physiological parameters of a user in real time; and
a processing unit configured for storing the physiological parameters, establishing a user health model comprising indicators of physiological parameters of the user in a healthy state according to stored physiological parameters, determining a level of current physiological parameters of the user according to the indicators of the physiological parameters of the user health model, and determining a physiological state of the user.

9. The system of claim 8, wherein the system further comprises an identification information verifying unit configured for obtaining identification information of the user; and
verifying whether the identification information of the user is consistent with identification information pre-stored in a database, wherein
upon a condition that the identification information of the user is consistent with identification information pre-stored in the database, acquiring the physiological parameters of the user, and
upon a condition that the identification information of the user is not consistent with identification information pre-stored in the database, creating new identification information of the user and acquiring the physiological parameters of the user.

10. The system of claim 8, wherein the processing unit is further configured for pre-processing of the physiological parameters and removing abnormal physiological parameters.

11. The system of claim 10, wherein the processing unit is further configured for smoothing and normalizing the physiological parameters.

12. The system of claim 8, wherein the processing unit is further configured for periodically updating the user health model according to the acquired physiological parameters of the user to obtain updated indicators of physiological parameters of the user in the healthy state.

13. The system of claim 8, wherein the physiological parameters comprise at least one of a pulse rate, a skin temperature, a skin resistance, or a step count.

14. The system of claim 8, wherein the processing unit is further configured for generating prompt information of physiological parameters being abnormal upon a condition that the current physiological parameters of the user exceed a range of the indicators of the physiological parameters.

15. The system of claim 8, wherein the physiological parameter acquiring unit comprises a wearable device, and/or the processing unit comprises a server.

16. The system of claim 8, wherein the processing unit is further configured for transmitting information of a current level of physiological parameters of the user and physical status of the user to a user terminal.
